(19) United States
(12) Patent Application Publication
(10) Pub. No.: US 2018/0153731 A1
(43) Pub. Date: Jun. 7, 2018

(54) HYGIENIC PROTECTOR FOR MALE URINATION

(71) Applicant: SOL A TESTA, S.L., Burriana (Castellon) (ES)

(72) Inventor: Javier LLOPIS AMOROS, Burriana (Castellon) (ES)

(21) Appl. No.: 15/577,075

(22) PCT Filed: May 26, 2016
(86) PCT No.: PCT/ES2016/000063

§ 371 Date: Nov. 27, 2017

(30) Foreign Application Priority Data
May 27, 2015 (US) U201500374

(51) Int. Cl. A61F 5/453; A61F 13/471

(52) U.S. Cl. CPC A61F 5/453; A61F 13/471

(57) ABSTRACT

Hygienic Protector for male urination which is a hollow tube of an absorbent and elastic, textile material that has one or more fins arranged on the surface outside of the pipe concerned. Its advantages are the following: don't need to wash your hands before or after urination, the be absorbent paper, collects the last drops so it stains do not originate in the underwear or pants and if the penis during urination is completely inside the cover, reduces splashes with which reduces the number of times that wash services and removed almost completely, the odor-causing.

The object of the present invention is a device by way of cover of the penis to ensure hygiene and cleaning during male urination, and behind it.

### BACKGROUND OF THE INVENTION

All men are facing different problems during urination, among which it can quote, dirty hands and need to wash them before or get dirty hands during the action, and not having the means to do so, or those droplets that dirty clothes inside or the pants after urinating or splashes occurring and causing so many marital disagreements.

The hygienic protector for male urination is a device easy manufacturing and low cost which solves the above drawbacks and contributes to the hygiene and cleanliness of the users and services.

The patent application US2006/0090250 shows a urinary urination device constituted by a simple sheet of flexible material, substantially flat, sensibly receptive to water, which can provide a guide for the urine to flow from the urethra to a receptacle opening located by below. The manufacturing material can be eliminated in sewer systems without clogging or damaging the systems. The preferred material of the invention are non-woven, lightweight sheets similar to conventional toilet paper and which have been modified to not be entirely wet in the urine. A preferred material is a nonwoven, wet, biodegradable web material similar to the exemplified in U.S. Pat. No. 4,755,421. The device of the invention can also be formed from a thin and lightweight paper which can be coated on the outside to be at least temporarily impermeable, but which is sufficiently biodegradable to allow it to empty to dispose of the urinary support once Has been used. The problem presented by the patent application US 2006/0090250 is that the provision of a flat sheet of flexible material to direct the urine does not ensure that urination is oriented correctly, as it can flow through any part of the sheet, since it depends a lot on folding the sheet of material, in such a way that it does not guarantee the user a total safety at the time of urinating, so that urination is safe it is necessary that the device present a form such as to allow orienting the flow of urine from the user's penis, this is achieved by means of the present invention, since it consists of a tube of cylindrical or frustoconical shape that adapts to the end of the penis and said end intended to be in contact with the glans is folded towards the interior of the tube and the portion of the tube folded inward has a plurality of circumferential folds ensuring that the direction of the urine either by a single route and this is not expelled by any part, in addition the present invention comprises one or more fins for its manipulation by the user which increases its reliability.

### DESCRIPTION OF THE INVENTION

The hygienic protector for male urination object of the present invention consists of a hollow tube of absorbent, elastic, textile material that has one or more fins arranged on the outer surface of the pipe concerned.

### BRIEF DESCRIPTION OF THE FIGURES

FIG. 1; shows a view in perspective of a first priority realization of the invention.

FIG. 2; shows a view in perspective of a second priority realization of the invention.

FIG. 3: shows a view in perspective of a third preferred realization of the invention.

### PREFERRED EMBODIMENT OF THE INVENTION

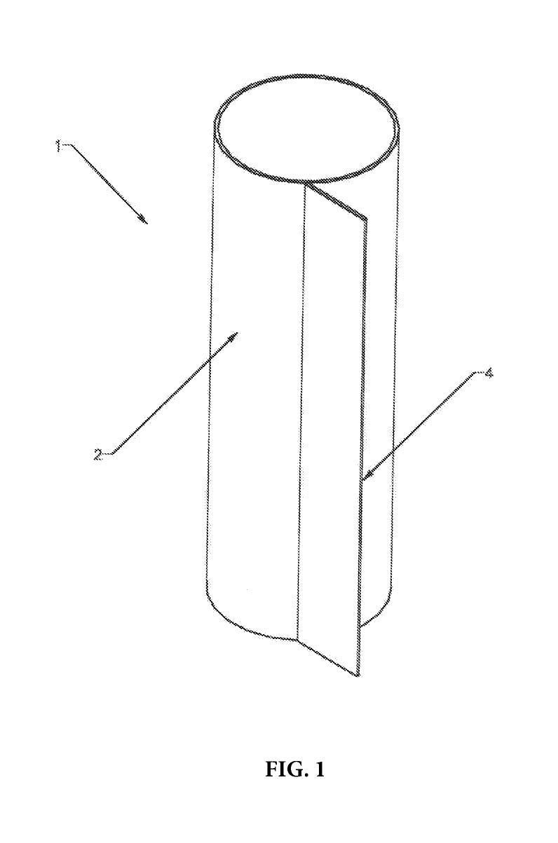

FIG. 1 shows a first priority realization of the hygienic protector for urination male, which consists in a Hollow tube, preferably approximately cylindrical shape, absorbent, elastic textile material, whose surface outside has one or more fins to facilitate handling.

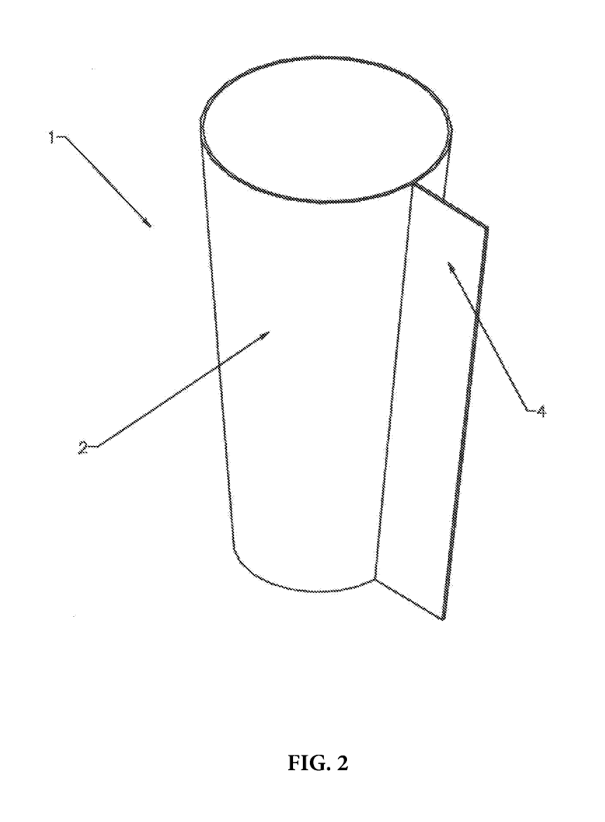

FIG. 2 shows a second preferred realization of the invention, which consists in a hollow tuve, preferably approximately Pan shape, of a material absorbent and elastic textile, preferably paper, whose outer surface has one or more fins to facilitate its manipulation.

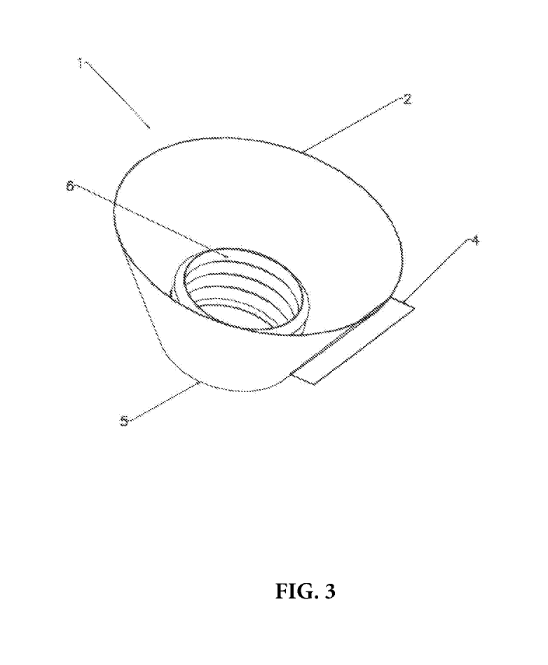

FIG. 3 shows a third preferred realization of the invention, which consists in a preferably hollow tube of Pan shape (2), an absorbent textile material and elastic, preferably paper, whose outer surface has one or more fins to facilitate handling. In addition, the end (5) destined to be in contact with the Glans is folded towards the inside of the pipe concerned and the portion of the tube folded inward can have a plurality of circumferential pleats. (6)

As a design option, the surface of the pipe (2) hole has a plurality of pleats, not shown in the figure, uniformly distributed to facilitate their adaptation to different sizes of penises.

The way to use the hygienic protector for male urination, which can come in boxes, is take one from the box, extend the folded portion of tube to inside, grab it by the flaps or stretch it with the fingers, insert the penis until the Glans protrudes approximately 1 cm, urinate, partially remove the invention, shaken to collect the last drops, remove it and dry the tip of the Glans and finally throw away it to the litter bin.

In order to improve the cleaning, remove odors and prevent diseases, the inside surface of the hollow tube may incorporate a cleansing cream for skin and mucous membranes or a disinfectant cream.

The advantages of the present invention include, but are not limited, it is not necessary to wash the hands before or after urination, being absorbent paper, collects the last drops so stains do not originate in the underwear or pants and if the penis during urination is completely inside the cover, reduces splashes with which reduces the number of times that wash services and removed almost completely, the odor-causing.

1. Hygienic Protector for male urination characterized that it consists of a hollow tube of an absorbent and elastic textile material that it has one or more fins arranged on the outer surface of the referred hollow tube.

2. Hygienic Protector for male urination, according to claim 1, it is characterized because the tube is approximately cylindrical shaped.

3. Hygienic Protector for male urination, according to the claim 1, characterized because the tube is truncated cone shape.

4. Hygienic Protector for male urination, according to any of the previous claims, characterized because the surface of the tube has of a plurality of evenly distributed longitudinal folds.

5. Hygienic Protector for male urination, according to any of the previous claims, characterized because in that the end of the tube intended to be in the glans is folded towards the inside of the tube.

6. Hygienic Protector for male urination, according to any of the previous claims, characterized wherein the part of the tube that is folded towards the inside of said tube has a plurality of circumferential folds.

7. Hygienic Protector for male urination, according to any of the previous claims, characterized because the textile material is paper.

8. Hygienic Protector for male urination, according to any of the preceding claims, characterized by that on the inside of the tube surface incorporates a cleansing cream for skin and mucous membranes.

9. Hygienic Protector for male urination, according to any of the above claims, characterized because in the inner surface of the tube incorporates a disinfectant cream.
